(19) United States
(12) Patent Application Publication
(10) Pub. No.: US 2024/0257992 A1
(43) Pub. Date: Aug. 1, 2024

(54) DEVICES AND METHODS FOR CONVEYING NEURON SIGNALS TO A PROCESSING DEVICE

(71) Applicant: University of Pittsburgh - Of the Commonwealth System of Higher Education, Pittsburgh, PA (US)

(72) Inventors: Andrew B. Schwartz, Pittsburgh, PA (US); May Yoon PWINT, Pittsburgh, PA (US)

(73) Assignee: University of Pittsburgh - Of the Commonwealth System of Higher Education, Pittsburgh, PA (US)

(21) Appl. No.: 18/578,660

(22) PCT Filed: Jul. 14, 2022
(86) PCT No.: PCT/US2022/037110

§ 371 Date: Jan. 11, 2024

Related U.S. Application Data
(60) Provisional application No. 63/222,004, filed on Jul. 15, 2021.

(51) Int. Cl. H01B 7/00; A61B 5/293; C23C 14/04; C23C 14/08; C23C 14/14; C23C 16/04; C23C 16/56; G06F 3/01; H01B 13/00

(52) U.S. Cl. CPC H01B 7/0009; A61B 5/293; C23C 14/042; C23C 14/081; C23C 14/14; C23C 16/04; C23C 16/56; G06F 3/015; H01B 13/0036; A61B 2562/222

(57) ABSTRACT

Disclosed herein is a method for forming a cable configured to convey neuron signals. The method can include forming two longitudinal halves of the cable and bonding the two longitudinal halves together. Each of the two longitudinal halves can be formed on a respective substrate and includes a plurality of groups of conductive traces each in a respective layer of the respective longitudinal half. Each respective group of the plurality of groups of conductive traces can formed by depositing a respective insulating layer, patterning the respective insulating layer, depositing a respective conductive layer over at least a portion of the patterned insulating layer, patterning the respective conductive layer into the respective group of conductive traces. Each respective layer of the respective longitudinal half can have a width smaller than a layer formed prior to the respective layer, and can have a thickness that is within a range of 2 microns and 10 microns.

### CROSS-REFERENCE TO RELATED APPLICATION

This application is related to and claims priority under 35 U.S. § 119(e) the U.S. Provisional Patent Application No. 63/222,004, filed Jul. 15, 2021, titled “DEVICES AND METHODS FOR CONVEYING NEURON SIGNALS TO A PROCESSING DEVICE,” the entire contents of which are incorporated herein by reference for all purposes.

### FIELD OF DISCLOSURE

The present disclosure is generally related to devices and methods of conveying neural signals, including but not limited to devices, systems and methods involving a cable to convey neuron signals to a processing device.

### BACKGROUND

Developments of remote-controlled devices and extended reality immersive technologies are complex technological fields. Devices such as prosthetics and mind-controlled robotics should be engineered to be mechanically capable of accomplishing the required task, but also incorporate or operate with a system which can determine a user's intent for the specific task. Similarly, immersive technologies should capture a user's intended action for application by an avatar within a virtual reality (VR), augmented reality (AR), or mixed reality environment. Accessing neuron signals to decode a user's intended action remain an area to be developed.

### SUMMARY

In certain embodiments, disclosed herein are systems, methods, and non-transitory computer readable medium for accessing and/or utilizing neuron signals or spike trains for decoding a subject's intended motion for instance.

In one aspect, a cable (e.g. interconnect or signal cable/connector/wire) is implemented or configured for conveying neuron signals (e.g., electrical signals triggered, translated, converted and/or amplified from spike trains, synapse impulses and/or other brain-related signals). In some embodiments, the cable includes a plurality of conductive traces or leads extending in a first direction/path (e.g., along a length or longitudinal direction of the cable). The cable can include a first dielectric material extending in the first direction/path and surrounding (or wrapping around) each of the plurality of conductive traces or leads in each direction perpendicular to the first direction/path; and a second dielectric material extending in the first direction/path and surrounding the first dielectric material in the each direction.

In some embodiments, an intracortical microelectrode array is coupled to the cable to convey neuron signals from a brain to the cable. The cable may convey the neuron signals to a processing device. The processing device may for instance be configured to determine that the neuron signals match or correlate to an appropriate movement or subject's intention, and/or may determine that the neuron signals indicate brain damage, regression or injury, etc.

In some embodiments, the plurality of conductive leads and the first dielectric material are arranged in alternating layers (e.g., planar or non-planar, such as concentric). In some embodiments, each layer is 2-10 microns thick. In some embodiments, each layer is 5 microns, or substantially near (e.g., within 5% of) 5 microns. The cable may be configured to convey the neuron signals for processing, to enable or facilitate at least one of: research, training of motor function, rehabilitation (of a damaged brain), treatment of brain related disease, injury or disorder, detection or diagnosis of brain damage, brain function disorder or brain injury, control of a robotic device, or an action or movement of a virtual entity.

In some embodiments, the plurality of conductive leads are composed of one or more of metal, alloy, platinum, (MP-35) stainless steel, gold, or iridium. In some embodiments, the first or second dielectric material is composed of one or more of polymer, liquid crystal polymer, parylene, or polyimide. In some embodiments, the first or second dielectric material comprises a water/moisture-proof and/or sealant material such as aluminum oxide (AL₂O₃). In some embodiments, the second dielectric material is composed of at least one of: silicone or rubber.

In another aspect, disclosed herein is a cable apparatus for conveying neuron signals. In some embodiments, the cable apparatus includes a cable housing a plurality of conductive leads/traces, each of the plurality of conductive leads/traces encased by one or more dielectric material/layers; and a terminal coupled to the cable, the terminal having a form factor that is configured to match a backplane of an array for processing neuron signals. The terminal can include a plurality of first terminals, each first terminal connecting/extending from a respective one of the conductive leads/traces.

The cable apparatus may include another terminal coupled to the plurality of conductive leads, the another terminal having a form factor corresponding to that of the terminal. The another terminal can include a plurality of second terminals, each second terminal connecting/extending from a respective one of the conductive leads/traces. The plurality of conductive leads may be configured to convey the neuron signals for processing, such as to enable or facilitate at least one of: research, training of motor function, rehabilitation, treatment of brain related disease, injury or disorder, detection or diagnosis of brain damage, brain function disorder or brain injury, control of a robotic device, or an action or movement of a virtual entity.

In yet another aspect, disclosed herein is a method for forming (e.g., fabricating or constructing) a cable configured to convey neuron signals. In some embodiments, the method includes forming alternating layers of insulation (or non-conductive) material and conductive material, wherein each layer of insulation material is formed using vapor or other deposition method; and each layer of conductive material is formed by depositing the conductive material; removing selected portions of the conductive material using subtractive processing; and filling trenches where the selected portions were removed with the insulation material. In some embodiments, the method further includes forming two halves of the cable, wherein each half of the two halves includes a bottom layer of the alternating layers that is the widest amongst the alternating layers, wherein each successive layer formed above the bottom layer is narrower than a layer on which it is formed; and bonding the two halves together.

In a further aspect, disclosed is a method which comprises receiving, by an interconnect cable from an intracortical microelectrode array, neuron signals. The interconnect cable may convey the neuron signals to a processing device, such as to enable or facilitate at least one of: research, training of motor function, rehabilitation, treatment of brain related disease, injury or disorder, detection or diagnosis of brain damage, brain function disorder or brain injury, control of a robotic device, or an action or movement of a virtual entity.

In another embodiment, the disclosure encompasses a method for training motor function in a subject in need. The method comprises chronically implanting a cable or cable apparatus (e.g., which may include the cable/interconnect and/or an intracortical microelectrode array) described herein, in the subject.

In another embodiment, the disclosure encompasses a method for rehabilitation in a subject in need. The method comprises chronically implanting a cable or cable apparatus described herein, in the subject.

In another embodiment, the disclosure encompasses a method for treatment of brain related disease, injury or disorder in a subject in need. The method comprises chronically implanting a cable or cable apparatus described herein, in the subject.

In another embodiment, the disclosure encompasses a method for detection or diagnosis of brain damage, brain function disorder or brain injury in a subject in need. The method comprises chronically implanting a cable or cable apparatus described herein, in the subject.

In another embodiment, the disclosure encompasses a method for control of a robotic device in a subject in need. The method comprises chronically implanting a cable or cable apparatus described herein, in the subject.

In another embodiment, the disclosure encompasses a method for control of an action or movement of a virtual entity associated with a subject in need. The method comprises chronically implanting a cable or cable apparatus described herein, in the subject.

For all of the methods comprising chronically implanting a cable or cable apparatus in a subject in need, implantation can occur in an area of the primary motor cortex.

Both the foregoing summary and the following description of the drawings and detailed description are illustrative and explanatory. They are intended to provide further details of the disclosure, but are not to be construed as limiting. Other objects, advantages, and novel features shall be readily apparent to those skilled in the art from the following detailed description of the disclosure.

### BRIEF DESCRIPTION OF THE DRAWINGS

FIG. 1 is a block diagram of a computing environment according to an example implementation of the present disclosure.

FIG. 2 is a cross-sectional view of a cable for conveying neuron signals, according to one or more disclosed embodiments.

FIG. 3 includes multiple embodiments of a cable for conveying neuron signals, according to one or more disclosed embodiments.

FIGS. 4A-4H are top views of an example cable 800 for conveying neuron signals, corresponding to various fabrication stages, in accordance with various embodiments.

FIGS. 5A-5C are top views of an example cable 800 for conveying neuron signals, corresponding to various fabrication stages, in accordance with various embodiments.

### DETAILED DESCRIPTION

### I. Overview

Neurons in the primary motor cortex (M1) can encode physical parameters of limb movements such as direction, force, extend, load, or posture, in neuron signals or spike trains. Such physical parameters are proposed to be encoded by the spikes and spike rates for instance. In the visual and somatosensory cortex, spike times can carry significant information. Data from visual sensory cortex show highly precise response of neurons to repeating stimuli across the hierarchy of visual processing from retina, to Lateral Geniculate Nucleus (LGN) up to the higher visual areas and can be considered as temporal coding in the visual cortex. Similar temporal dynamics can be found in the auditory cortex vestibular system and somatosensory cortex. As used herein, the terms “spike train” or “neural spike train” refer to a sequence of spikes or action potentials with respect to time. Spikes, also known as action potentials, refers to a change in the voltage across a neuron membrane due to the flow of ions into and out of the neuron.

The motor cortex can be seen as similar or analogous to the somatosensory cortex in that it is in the same direct contact with the world as the somatosensory system is. For instance, the limb motion is expected to be associated with the same precise neural response as the neural response of a skin or joint encoding neuron would be expected to have. In view of the above considerations and applications for example, systems and methods for accessing and conveying/communicating neuron signals to processing systems are thus beneficial.

### IV. Cable for Conveying Neuron Signals

Disclosed herein are embodiments of a cable, also sometimes referred to as a micro-interconnect cable (MIC). The micro-interconnect cable (MIC) is designed, implemented and/or configured to connect intracortical microelectrode arrays to devices used to transmit/convey and/or process recorded brain signals. In some implementations, an interconnect for this purpose may include individual wires bound together with elastomeric adhesive. However, such an interconnect may be relatively large, stiff, and prone to insulation failure at points of connection.

Embodiments of the MIC and methods for fabricating the MIC disclosed herein can use planar (or layer-based fabrication) technology to make a three-dimensional (3D) object. Embodiments of the MIC and methods disclosed herein can provide flexible, small, hermetic means of conveying signals such as brain/neuron signals. The cable can be part of, or coupled to, an intracortical microelectrode array or other device suitable for implantation at least partially inside a body of a human or other animal. The MIC and the associated device can be implanted in, or attached to, the brain, a primary motor cortex of the brain, the heart, the central or peripheral nervous system, or anywhere else in the body.

In some embodiments, the MIC design includes one or more of the following characteristics. In some embodiments, the cable is flexible (e.g., 10-500 kPa). In some embodiments, a length of the cable is 4-7 cm, or any of other various values/ranges (e.g., 10-20 cm, or 20-50 cm). In some embodiments, a total cable thickness is 2-4 mm, or any of other various values (e.g., 0.5-1 mm, or 1-5 mm), with a protective coating of silicone and/or rubber, or any of other various insulation materials. In some embodiments, a cross-sectional profile of the cable is round, square, rectangular, triangular, or elliptical in shape, or any of other various profile shapes.

In some embodiments, the cable is composed of multiple layers of alternating insulator (e.g., substrate, insulation material, dielectric material) and conductor (e.g., conductive material, metallic material). In some embodiments, the number of layers can be between 10 and 40, or any of other various values. In some embodiments, each layer has a thickness of less than 1 micron, 1-5 microns, or 5-10 microns, 2-10 microns, 5 microns, substantially near 5 microns, or any of other various thickness values. In some embodiments, the thickness of the layer may depend on one or more of a type of polyimide used or spin speed. In some embodiments, the insulator (which can include multiple layers or components around parts of the conductor) includes polymer, liquid crystal polymer, parylene, polyimide, Aluminum oxide, or any of other various insulation and/or organic/polymer materials.

In some embodiments, the conductor includes at least one metal or alloy, such as platinum, (e.g., MP-35) stainless steel, gold, platinum, iridium, or any of other various conductor materials. The conductor may be implemented as a number of leads/traces (e.g., conductive leads) that extend in the same direction (e.g., straight or non-straight path) as the cable. In some embodiments, each cable contains 100-1000 signal leads/traces (or other ranges, such as 50-500, or 200-5000). In some embodiments, each lead/trace is comprised of the conductive material and is at least part of one of the number of conductive layers. In some embodiments, each lead is approximately 0.02 inches wide, or any of other various widths (e.g., 0.2-5 microns). In some embodiments, a suitable dielectric may be disposed around each lead to mitigate channel crosstalk or interference. In some embodiments, a suitable dielectric (e.g., sealant/waterproof layer), e.g., aluminum oxide, may be disposed around each lead/trace to mitigate water transgression. In some embodiments, subtractive processing (e.g., etching, isotropic etching, anisotropic etching, reactive ion etching, neutral beam etching, or any other process of subtractive processing) is used to remove metal (e.g., a portion of the conductor) to leave other portion(s) to form the leads in each layer, or any of other various processes.

In some embodiments, a cable terminal of the cable has a form factor that matches a backplane of an array such as an intracortical microelectrode array (e.g., Utah electrode array). In some embodiments, the backplane of the array is 4×4 mm, or any of other various values, with a pattern of 10×10 gold/conductive contacts, or any of other various arrangement of various types of contacts. In some embodiments, the terminal has through-holes to match the array (e.g., UEA) form factor. In some embodiments, ultrasonic bonding is used to attach the terminal to the array, or any of other various bonding/coupling/connecting processes. In some embodiments, deposition (e.g., vapor deposition, ion-beam deposition, sputtering, spin coating, electroplating, oxidizing, or any other process of depositing) of a suitable insulator (e.g. Parylene) is used to cover and hermetically seal the lead/trace, terminal and/or array connection. In some embodiments, the terminal end of the cable is bifurcated/branched/split to accommodate (or couple to) two or more intracortical microelectrode arrays (e.g., 100-electrode UEAs). In some embodiments, an opposite end (e.g., a second terminal end opposite the cable from the terminal end) of the cable is fabricated to specification for an appropriate next-stage, downstream recording, and/or processing device.

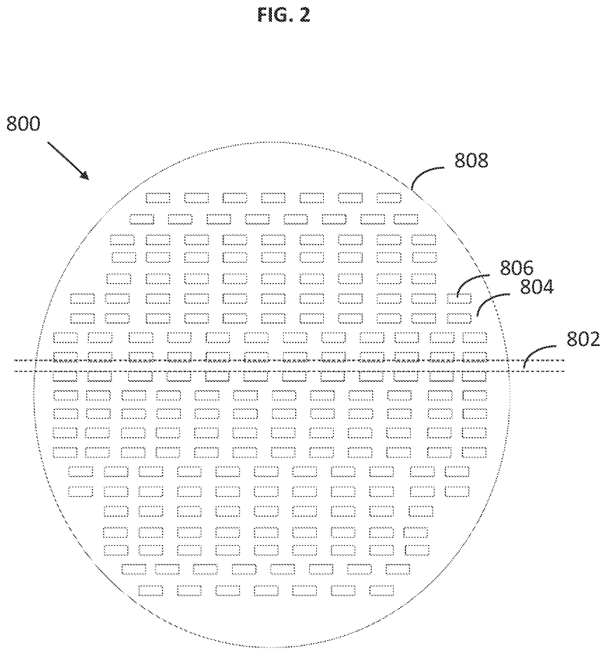

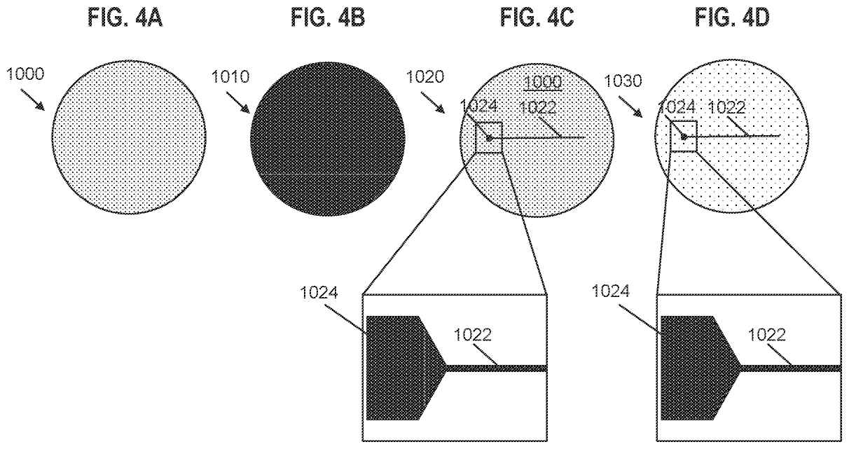

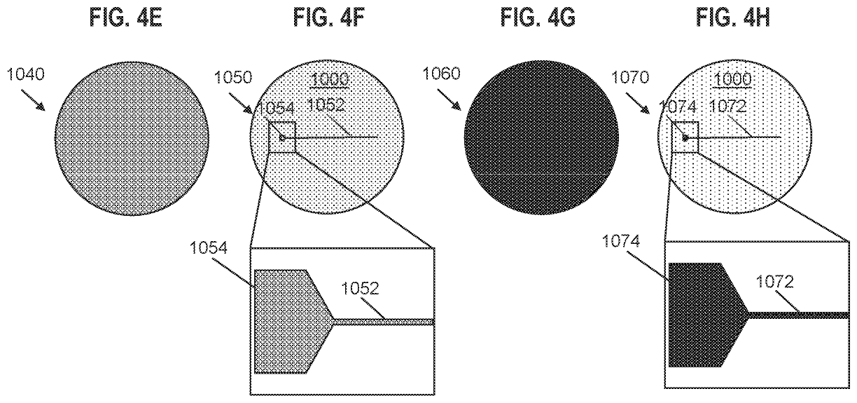

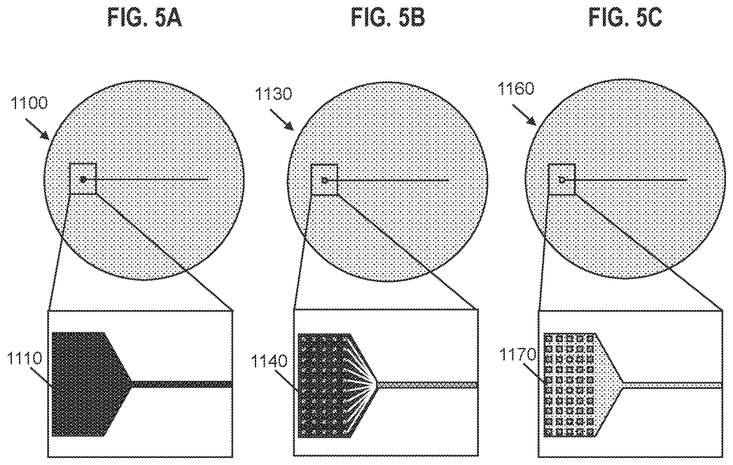

FIG. 2 is a cross-sectional view of the cable 800 (e.g., MIC, cable portion of the MIC, microfabricated device) for conveying the neuron signals, according to one or more disclosed embodiments. Layers can be photo deposited or printed. Lithographic techniques can be used to deposit individual layers 802. In some embodiments, each layer 802 is composed/comprised of insulating material 804 (e.g., insulator, insulation/dielectric material, a polymer, polyimide, or liquid crystal polymer substrate). In some embodiments, disposed (e.g., sandwiched) between the layers 802 of insulating material 804 are conductors 806 (e.g., metal leads). The conductors can be stainless steel, platinum, or platinum/iridium. An outline 808 (e.g., cross-sectional profile/shape) of the cable can be round. To accomplish a round (or other) outline 808, the device may be fabricated in halves (or other portions or segments) with the widest layer of the layers 802 on the bottom (or first) and successive layers of the layers 802 having smaller widths/diameters. The halves can then be bonded (e.g., on the widest sides) to form the cylindrical structure of the cable. In one example, a cross-section of the cable 800 can have approximately 20 or more leads and the diameter can be less than 0.5 mm for instance. More detail is shown with respect to FIGS. 4A-4H and 5A-5C.

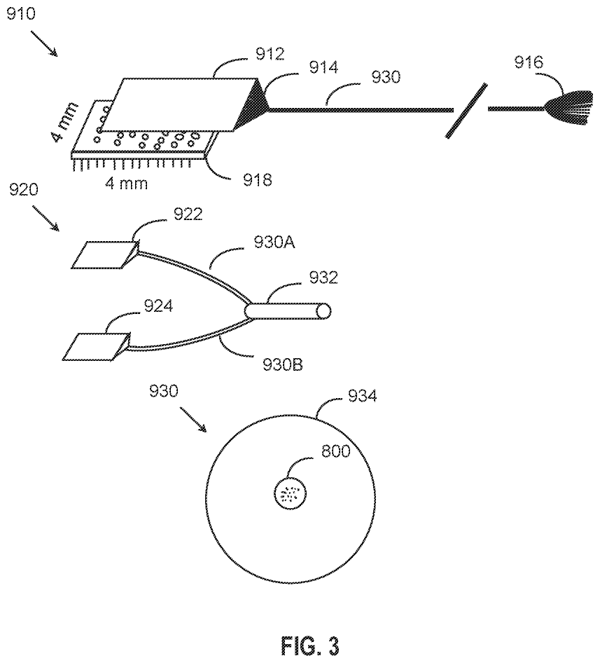

FIG. 3 includes multiple embodiments of a cable 800 for conveying neuron signals, according to one or more disclosed embodiments. The cable apparatus 910 includes a cable body 930, a terminal 912, a fan-out 914, and/or a terminal 916 opposite the cable body 930 from the terminal 912. The terminal 912 can be a square (or rectangular or other shape), planar interconnect terminal. In some embodiments, the terminal 912 is 4 mm×4 mm. The terminal 912 can match the form factor of an array such as the Utah array 918. The fan-out 914 can include one or more conductors such as the conductor 806 extending from the cable 800 to the terminal 912. Each conductor may be referred to as a lead or a conductive lead. The conductor 806 may be referred to as a lead 806, a conductive lead 806, a trace 806, or a conductive trace 806. The terminal 916 may have a fan-out similar to the fan-out 914 or a geometric form factor similar to the terminal 912 for connecting to a downstream device.

In some embodiments, the cable apparatus 920 includes two terminals 922 and 924, and two cable bodies 930A and 930B, and a dual-cable body 932. The cable body 930A may couple the dual-cable body 932 (e.g., a first portion of conductors, each conductor similar to the conductor 806) to the terminal 922 and the cable body 930B may couple the dual-cable body 932 (e.g., a second portion of conductors, each conductor similar to the conductor 806) to the terminal 924. In the example, two arrays similar to the Utah array 918 can connect to (e.g., share) the same dual-cable body 932 via the terminals 922 and 924.

The cable body 930 can include an insulator 932 and the cable 800. The insulator 932 can be silicone and/or rubber. The insulator 932 can encase (e.g., surround, wrap around) the cable 800. The insulator 932 may be used for mechanical protection of the cable 800. The cable can be a flexible round, micro-cable with approximately 200 leads and a diameter of approximately 0.5 mm, as one example. The cable can connect intracortical microelectrode arrays to external or internal termination sites with minimal tethering forces. The design of the cable can make it possible to hermetically seal all connections in and to the cable.

FIGS. 4A-4H are top views of an example cable 800 for conveying neuron signals during various fabrication stages, in accordance with various embodiments. In some embodiments, the cable 800 is formed using a planar technology, although the fabrication process described in FIGS. 4A-4H can be applied to any three-dimensional (3D) object without departing from the scope of the present disclosure.

FIG. 4A is a perspective view of the substrate 1000 upon which the example cable 800 for conveying neuron signals is formed at one of the various stages of fabrication, in accordance with various embodiments. The substrate 1000 may be a semiconductor substrate, such as a bulk semiconductor, a semiconductor-on-insulator (SOI) substrate, or the like, which may be doped (e.g., with a p-type or an n-type dopant) or undoped. The substrate 1000 may be a wafer, such as a silicon wafer. Generally, an SOI substrate includes a layer of a semiconductor material formed on an insulator layer. The insulator layer may be, for example, a buried oxide (BOX) layer, a silicon oxide layer, or the like. The insulator layer is provided on a substrate, typically a silicon or glass substrate. Other substrates, such as a multi-layered or gradient substrate may also be used. In some embodiments, the semiconductor material of the substrate 1000 may include silicon; germanium; a compound semiconductor including silicon carbide, gallium arsenic, gallium phosphide, indium phosphide, indium arsenide, and/or indium antimonide; an alloy semiconductor including SiGe, GaAsP, AlInAs, AlGaAs, GalnAs, GaInP, and/or GaInAsP; or combinations thereof. Other materials are within the scope of the present disclosure.

FIG. 4B is a top view of the insulating layer 1010 which is formed at one of the various stages of fabrication, in accordance with various embodiments. The insulating layer 1010 may be formed by depositing the insulating layer over the substrate 1000. The insulating layer 1010 may include at least one of polyimide, polyimide precursor, polymer, liquid crystal polymer, or parylene, although other various plastics and/or other insulating/dielectric materials may be used while remaining in the scope of the present disclosure. In some embodiments, the insulating layer 1010 is photo-sensitive and may include or be formed using a photoresist. The insulating layer 1010 can be deposited by a conformal deposition method such as chemical vapor deposition (CVD), or by a self-planarizing deposition (SPD) process such as spin coating. The chemical vapor deposition may include a low-pressure chemical vapor deposition (LPCVD). Other methods of depositing the dielectric fill material 630 are within the scope of the present disclosure. Optionally, a chemical mechanical planarization (CMP) process may be performed to planarize the insulating layer remove any excess insulating material.

FIG. 4C is a top view of the patterned insulating layer 1020 which is formed at one of the various stages of fabrication, in accordance with various embodiments. The patterned insulating layer 1020 may be formed by patterning the insulating layer 1010. The patterned insulating layer 1010 can be patterned by using a mask aligner or a maskless laser direct writing system. The insulating layer 1010 can be patterned by using a photomask and etching the insulating layer 1010. The insulating layer 1010 can be etched using a reactive ion etcher, although other etching processes such as neutral beam etching are within the scope of the present disclosure. In some embodiments, the portions of the insulating layer 1010 that are removed by etching are the portions of the insulating layer 1010 exposed by the photomask. In some embodiments, the portions of the insulating layer 1010 that are removed by etching are the portions of the insulating layer 1010 not exposed (e.g., covered) by the photomask. The patterned insulating layer 1020 can be cured (e.g., in an oxygen-free environment), e.g., into structure conformed or aligned with the conductor traces to be formed later. The curing may be performed at temperatures within the range of 300 degrees Celsius to 400 degrees Celsius, or at other various temperatures.

The patterned insulating layer 1020 may include a body portion 1022 and a terminal portion 1024. The body portion 1022 and the terminal portion 1024 may be formed on top of the substrate 1000. The terminal portion may include insulation for a terminal similar to the terminal 912 and a fan-out similar to the fan-out 914. The body portion 1022 may contain insulation for a plurality of a group of leads (or traces), wherein each lead is similar to the lead 806.

FIG. 4D is a top view of a photoresist 1030 which is formed at one of the various stages of fabrication, in accordance with various embodiments. The photoresist 1030 may be used for metal patterning. The photoresist 1030 may be deposited over the patterned insulating layer 1020 and the substrate 1000. The photoresist 1030 may be referred to as a photoresist mask 1030, a positive photoresist 1030, positive photoresist mask 1030, a sacrificial layer 1030, a sacrificial photoresist mask 1030, or a sacrificial positive photoresist mask 1030. Non-limiting examples of the photoresist 1030 include silicon nitride, an amorphous semiconductor material (such as amorphous silicon), or a polycrystalline semiconductor material (such as polysilicon).

FIG. 4E is a top view of a conductive layer 1040 which is formed at one of the various stages of fabrication, in accordance with various embodiments. The conductive layer 1040 may be formed by depositing the conductive layer 1040. The conductive layer 1040 may be deposited over the photoresist 1030. The conductive layer 1040 can be deposited by using electron-beam vapor deposition (EBPVD), although other types of deposition such as sputtering, ion beam sputtering, CVD, LPCVD, or SPD are within the scope of the present disclosure. The conductive layer 1040 may include at least one of gold, platinum, stainless steel, iridium or other metal or alloy.

FIG. 4F is a top view of a patterned conductive layer 1050 (e.g., comprising a plurality of conductive leads/traces, and/or corresponding terminals) which is formed at one of the various stages of fabrication, in accordance with various embodiments. The patterned conductive layer 1050 may be formed by patterning the conductive layer 1040. The conductive layer 1040 can be patterned by removing portions of the conductive layer 1040 and the photoresist 1030 using acetone for instance. In some embodiments, the portions of the conductive layer 1040 that are removed may be the portions under which the photoresist 1030 is deposited directly over the substrate 1000.

The patterned conductive layer 1050 may include a body portion 1052 and a terminal portion 1054. The body portion 1052 and the terminal portion 1054 may be formed on top of the body portion 1022 and the terminal portion 1024, respectively. The terminal portion 1054 may include leads for a terminal similar to the terminal 912 and a fan-out similar to the fan-out 914. The body portion 1052 may contain a plurality of a group of leads (or traces), wherein each lead is similar to the lead 806.

FIG. 4G is a top view of a coating 1060 which is formed at one of the various stages of fabrication, in accordance with various embodiments. The coating 1060 may be used to mitigate water transgression. The coating 1060 may comprise aluminum oxide or some other insulating/(e.g., sealing, sealant, water-proof) material. The coating 1060 may comprise a similar material to the insulating layer 1010. The coating 1060 can be referred to as a waterproof coating 1060 or a sealant 1060. The coating 1060 can be formed by depositing the coating 1060. The coating 1060 can be deposited on top of the patterned conductive layer 1050 and the substrate 1000. The coating 1060 may be deposited using one of the deposition techniques described above. The coating 1060 may be deposited similarly to how the insulating layer 1010 is deposited. The coating 1060 may be deposited by placing a sacrificial mask over the patterned conductive layer 1050 and the substrate 1000, or only over the substrate 1000 (e.g., over the non-conductive portions of the wafer) and sputtering, or otherwise depositing, the coating layer 1060 onto the patterned conductive layer 1050, or onto the sacrificial mask.

FIG. 4H is a top view of a patterned coating 1070 which is formed at one of the various stages of fabrication, in accordance with various embodiments. The patterned coating 1070 may be formed by patterning the coating 1060. The coating 1060 may be patterned using one of the patterning techniques described above. The coating 1060 may be patterned similarly to how the insulating layer 1010 is patterned. The coating 1060 may be patterned by removing the sacrificial mask and/or portions of the conductive layer sputtered, or otherwise deposited, e.g., over the sacrificial mask.

The patterned coating 1070 may include a body portion 1072 and a terminal portion 1074. The body portion 1072 and the terminal portion 1074 may be formed on top of the body portion 1052 and the terminal portion 1054, respectively. The terminal portion 1074 may include coating for a terminal similar to the terminal 912 and a fan-out similar to the fan-out 914. The body portion 1072 may contain coating for a plurality of a group of leads, wherein each lead is similar to the lead 806.

In some embodiments, the stages depicted by one or more of the FIGS. 4B-4H are repeated such that a longitudinal half is formed. Although one longitudinal half is represented in the figures at different fabrication stages, this is for simplified illustration and not intended to be limited. For example, multiple longitudinal halves can be fabricated concurrently on the same substrate 1000, and these may be aligned spatially next to each other on the substrate 1000 for instance. A longitudinal half can include alternating layers of conductive layers and insulating layers. Each conductive layer can include groups of leads formed in accordance with FIGS. 4D-4F. Each respective layer of the longitudinal half can have a width smaller than a layer formed prior to the respective layer. Each respective layer can have a thickness that is less than 1 micron, 5 microns, 15 microns, in a range of 2 microns and 10 microns, or some other value. In some embodiments, two such longitudinal halves are formed, for pairing. In some embodiments, the two longitudinal halves are bonded together to form the cable 800.

FIGS. 5A-5C are top views of an example cable 800 for conveying neuron signals during various fabrication stages, in accordance with various embodiments. Any of the fabrication stages of FIGS. 5A-5C can be combined with any of the fabrication stages of FIGS. 4A-4H.

FIG. 5A is a top view of a first insulating layer 1100 which is formed at one of the various stages of fabrication, in accordance with various embodiments. The first insulating layer 1100 includes the terminal layer 1110, which can be the terminal portion 1024 and/or an insulating layer of the terminal 912. The terminal layer 1110 can be formed by depositing and patterning an insulating layer similarly to how the insulating layer 1010 is deposited and patterned in FIGS. 4B-4C.

FIG. 5B is a top view of a conductive layer 1130 which is formed at one of the various stages of fabrication, in accordance with various embodiments. The conductive layer 1130 includes the terminal layer 1140, which can be the terminal portion 1054 and/or a conductive layer of the terminal 912. The terminal layer 1130 can be formed by depositing and patterning a conductive layer similarly to how (or as part of) the conductive layer 1050 is deposited and patterned in FIGS. 4D-4F.

FIG. 5C is a top view of a second insulating layer 1160 which is formed at one of the various stages of fabrication, in accordance with various embodiments. The second insulating layer 1160 includes the terminal layer 1170, which can be the terminal portion 1074 and/or a coating layer of the terminal 912. The terminal layer 1170 can be formed by depositing and patterning a coating layer similarly to how (or as part of) the coating layer 1070 is deposited and patterned in FIGS. 4G-4H.

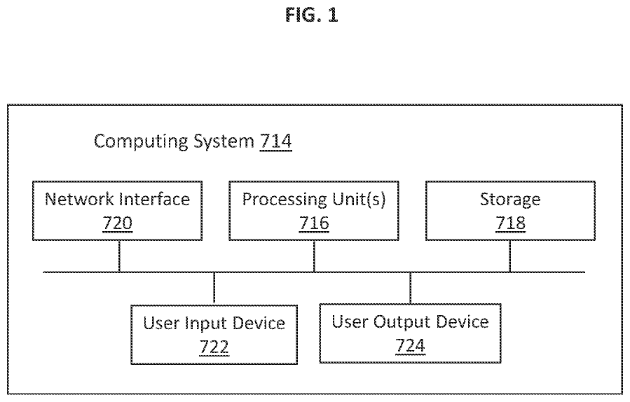

Various operations described herein can be implemented on one or more computer systems. FIG. 1 shows a block diagram of a representative computing system 714 usable to implement the present disclosure. The computing system 714 can be a processing system to which a cable apparatus is connected to, or a system managing/controlling the fabrication processes discussed herein. In some embodiments, the system 200 is implemented by the computing system 714. Computing system 714 can be implemented, for example, as a consumer device such as a smartphone, other mobile phone, tablet computer, wearable computing device (e.g., smart watch, eyeglasses, head mounted display), desktop computer, laptop computer, cloud computing service or implemented with distributed computing devices. In some embodiments, the computing system 714 can include computer components such as processors 716, storage device 718, network interface 720, user input device 722, and user output device 724.

Network interface 720 can provide a connection to a wide area network (e.g., the Internet) to which WAN interface of a remote server system is also connected. Network interface 720 can include a wired interface (e.g., Ethernet) and/or a wireless interface implementing various RF data communication standards such as Wi-Fi, Bluetooth, or cellular data network standards (e.g., 3G, 4G, 5G, 60 GHz, LTE, etc.).

User input device 722 can include any device (or devices) via which a user can provide signals to computing system 714; computing system 714 can interpret the signals as indicative of particular user requests or information. User input device 722 can include any or all of a keyboard, touch pad, touch screen, mouse or other pointing device, scroll wheel, click wheel, dial, button, switch, keypad, microphone, sensors (e.g., a motion sensor, an eye tracking sensor, etc.), and so on.

User output device 724 can include any device via which computing system 714 can provide information to a user. For example, user output device 724 can include a display to display images generated by or delivered to computing system 714. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). A device such as a touchscreen that function as both input and output device can be used. Output devices 724 can be provided in addition to or instead of a display. Examples include indicator lights, speakers, tactile “display” devices, printers, and so on.

Some implementations include electronic components, such as microprocessors, storage and memory that store computer program instructions in a non-transitory computer readable storage medium. Many of the features described in this specification can be implemented as processes that are specified as a set of program instructions encoded on a computer readable storage medium. When these program instructions are executed by one or more processors, they cause the processors to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processor 716 can provide various functionality for computing system 714, including any of the functionality described herein as being performed by a server or client, or other functionality associated with message management services.

It will be appreciated that computing system 714 is illustrative and that variations and modifications are possible. Computer systems used in connection with the present disclosure can have other capabilities not specifically described here. Further, while computing system 714 is described with reference to particular blocks, it is to be understood that these blocks are defined for convenience of description and are not intended to imply a particular physical arrangement of component parts. For instance, different blocks can be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Implementations of the present disclosure can be realized in a variety of apparatus including electronic devices implemented using any combination of circuitry and software.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

### V. Definitions

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements. Technical and scientific terms used herein have the meanings commonly understood by one of ordinary skill in the art, unless otherwise defined. Any suitable materials and/or methodologies known to those of ordinary skill in the art can be utilized in carrying out the methods described herein.

Systems and methods described herein may be embodied in other specific forms without departing from the characteristics thereof. As used herein, “approximately,” “about” “substantially” or other terms of degree will be understood by persons of ordinary skill in the art and will vary to some extent on the context in which it is used. If there are uses of the term which are not clear to persons of ordinary skill in the art given the context in which it is used, references to “approximately,” “about” “substantially” or other terms of degree shall include variations of +/−10% from the given measurement, unit, or range unless explicitly indicated otherwise.

Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements may differ according to other exemplary embodiments, and that such variations are intended to be encompassed by the present disclosure.

As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. In certain exemplary embodiments, a subject is a human.

As used herein, the terms “subject” and “user” are used interchangeably.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. For example, the term “a cell” includes a plurality of cells, including mixtures thereof.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method for forming a cable configured to convey neuron signals, the method comprising:
forming two longitudinal halves of the cable; and
bonding the two longitudinal halves together;
wherein each of the two longitudinal halves is formed on a respective substrate and includes a plurality of groups of conductive traces each in a respective layer of the respective longitudinal half, and each respective group of the plurality of groups of conductive traces is formed by:
depositing a respective insulating layer;
patterning the respective insulating layer;
depositing a respective conductive layer over at least a portion of the patterned insulating layer; and
patterning the respective conductive layer into the respective group of conductive traces,
wherein each respective layer of the respective longitudinal half has a width smaller than a layer formed prior to the respective layer, and has a thickness that is less than 15 microns.

2. The method of claim 1, wherein the insulating layer comprises at least one of:
polyimide, polymer, liquid crystal polymer, or parylene.

3. The method of claim 1, wherein depositing the insulating layer includes spin coating the insulating layer.

4. The method of claim 1, wherein depositing the insulating layer includes chemical vapor depositing the insulating layer.

5. The method of claim 1, wherein patterning the insulating layer includes patterning the insulating layer using a mask aligner or a maskless laser direct writing system and curing the insulating layer in an oxygen-free environment.

6. The method of claim 1, wherein patterning the insulating layer includes patterning the insulating layer using a photomask and etching the insulating layer using a reactive ion etcher.

7. The method of claim 1, wherein the conductive layer comprises at least one of: gold, platinum, stainless steel, or iridium.

8. The method of claim 1, wherein patterning the conductive layer comprises:
patterning a photoresist mask;
depositing the conductive layer over at least the patterned photoresist mask; and
removing the photoresist mask, and portions of the conductive layer deposited over the photoresist mask.

9. The method of claim 8, wherein the photoresist mask is removed using acetone.

10. The method of claim 1, comprising depositing and patterning a coating layer on the conductive layer.

11. The method of claim 10, wherein the coating layer comprises aluminum oxide.

12. The method of claim 10, wherein depositing and patterning the coating layer comprises:
placing a sacrificial mask;
sputtering the coating layer onto the conductive layer; and
removing the sacrificial mask, and portions of the conductive layer sputtered over the sacrificial mask.

13. The method of claim 1, comprising forming a cable terminal at one end of one of the two longitudinal halves of the cable.

14. The method of claim 13, wherein forming the cable terminal comprises:
depositing the respective insulating layer;
patterning the respective insulating layer;
depositing the respective conductive layer; and
patterning the respective conductive layer to form a conductive trace with the cable terminal at one end of the conductive trace.

15. A cable configured to convey neuron signals, the cable comprising:
two longitudinal halves bonded together;
wherein each of the two longitudinal halves is formed on a respective substrate and includes a plurality of groups of conductive traces each in a respective layer of the respective longitudinal half, and
wherein each respective layer of the respective longitudinal half has a width smaller than a layer formed prior to the respective layer, and has a thickness that is within a range of 2 microns and 10 microns.

16. The cable of claim 15, wherein an intracortical microelectrode array is coupled to the cable to convey neuron signals from at least one region of a brain to the cable.

17. The cable of claim 15, wherein the cable is configured to convey the neuron signals to a processing device, wherein if the processing device determines that the neuron signals do not match an appropriate movement, the processing device determines that the neuron signals indicate brain damage, brain function disorder or brain injury.

18. The cable of claim 15, wherein the cable is configured to convey the neuron signals for processing to enable or facilitate:
research,
training of motor function,
rehabilitation,
treatment of brain related disease, injury or disorder,
detection or diagnosis of brain damage, brain function disorder or brain injury,
control of a robotic device, or
an action or movement of a virtual entity.

19. The cable of claim 15, wherein the insulating layer comprises at least one of:
polyimide, polymer, liquid crystal polymer, or parylene.

20. The cable of claim 15, wherein the conductive layer comprises at least one of:
gold, platinum, stainless steel, or iridium.

21-27. (canceled)
